(19) United States
(12) Patent Application Publication
(10) Pub. No.: US 2024/0158423 A1
(43) Pub. Date: May 16, 2024

(54) TWO-COMPARTMENT BIPOLAR MEMBRANE ELECTRODIALYSIS OF SALTS OF AMINO ACIDS

(71) Applicant: MONSANTO TECHNOLOGY LLC, Saint Louis, MO (US)

(72) Inventors: Justin Robert STRUBLE, Ballwin, MO (US); Jian XU, Wildwood, MO (US)

(21) Appl. No.: 18/279,989

(22) PCT Filed: Mar. 2, 2022
(86) PCT No.: PCT/US2022/018580

§ 371 Date: Sep. 1, 2023

Related U.S. Application Data
(60) Provisional application No. 63/156,583, filed on Mar. 4, 2021.

(51) Int. Cl. C07F 9/38; C07C 227/18; C07C 227/42

(52) U.S. Cl. CPC C07F 9/3813; C07C 227/18; C07C 227/42

(57) ABSTRACT

This disclosure relates to an improved electrodialysis method for preparing an amino acid (e.g., iminodiacetic acid) from a salt of the amino acid (e.g., disodium iminodiacetic acid) utilizing a two-compartment bipolar membrane electrodialysis process wherein at least a portion of the salt product stream comprising the amino acid and one or more salt thereof is recirculated to the two-compartment bipolar membrane. The process also comprises removing at least a portion of the recirculation stream and phosphonomethylating the amino acid therein. The process further comprises recovering a base product stream and utilizing the base product stream for preparing the salt of the amino acid.

### CROSS-REFERENCE TO RELATED APPLICATION

This application claims the benefit of, and priority to, U.S. Provisional Application No. 63/156,583, filed on Mar. 4, 2021. The entire disclosure of the above application is incorporated herein by reference.

### FIELD

This disclosure relates to an improved electrodialysis method for preparing an amino acid (e.g., iminodiacetic acid) from a salt of the amino acid (e.g., disodium iminodiacetic acid) utilizing a two-compartment bipolar membrane electrodialysis process wherein at least a portion of the salt product stream comprising the amino acid and one or more salt thereof is recirculated to the two-compartment bipolar membrane. The process also comprises removing at least a portion of the product stream or recirculation stream and phosphonomethylating the amino acid therein. The process further comprises recovering a base product stream and utilizing the base product stream for preparing the salt of the amino acid.

### BACKGROUND

Bipolar membrane electrodialysis (BME) enables production of an inorganic or organic acid from an inorganic or organic salt, respectively, by water splitting, which provides the protons for the acid formation. Bipolar membranes are capable of splitting water directly into H⁺ and OH⁻ ions without the formation of gasses such as H₂ or O₂. In a bipolar membrane electrodialysis process, the H⁺ and OH⁻ ions generated by water splitting in the interfacial region of the membrane migrate under the influence of an electric field to the cathode and anode, respectively. A two-compartment BME cell typically includes a bipolar membrane (BPM) and cation exchange membrane (CEM). For purposes of scale, typically multiple repeating units of BPM-CEM-BPM or CEM-BPM-CEM are placed between two electrodes thereby forming a two-compartment BME cell containing multiple base and salt compartments.

Generally, an electrodialysis process requires suitable ion conductivity to achieve a commercially acceptable current efficiency. Salts that dissociate well within the salt compartment are able to maintain sufficient ion conductivity and acceptable current efficiency. Where the salt is unable to achieve the required dissociation, it may be necessary to modify the process. For example, heat may be introduced into the process or a further ion exchange resin may be installed within the salt compartment of the bipolar membrane apparatus.

An electrodialysis process utilizing a two-compartment bipolar membrane apparatus wherein an amino acid is produced under improved and commercially acceptable current efficiencies would serve a need in the art including by, for example, eliminating the need to introduce heat into the process, eliminating the need for installation of a further ion exchange resin within the acid compartment of the bipolar membrane apparatus, and/or avoiding the generation of chloride-containing process streams.

### SUMMARY

Provided herein is a two-compartment bipolar membrane electrodialysis apparatus and process for improved production of an amino acid from a salt of the amino acid, wherein the process results in commercially acceptable current efficiencies and commercially acceptable yields of amino acid.

The present disclosure includes two-compartment bipolar membrane electrodialysis processes where the base product of the two-compartment bipolar membrane is substantially chloride free. When chloride is present in the base product, and subsequently used to prepare the salt of the amino acid, the catalyst used in preparing the salt of the amino acid may be prone to deactivation and/or poisoning because of the presence of chlorides. The processes of the present disclosure allow for a substantially chloride free base product, which aids in integration of the electrodialysis methods of the present disclosure with preparation of the amino acid salt. For example, the process of the present disclosure results in a base product having a chloride content of less than 200 ppm. More generally, the processes of the present disclosure are currently believed to provide improved process efficiency and commercially acceptable yields of the desired amino acid.

Briefly, therefore, the present disclosure is directed to a process for preparing iminodiacetic acid. The process comprises introducing a feed salt stream comprising disodium iminodiacetic acid (DSIDA) into a salt compartment of a two-compartment electrodialysis bipolar membrane cell comprising the salt compartment and a base compartment. A salt product stream is recovered from the salt compartment of the two-compartment bipolar membrane cell, the salt product stream comprising iminodiacetic acid (IDA) and monosodium iminodiacetic acid (MSIDA). A base product stream is recovered from the base compartment of the two-compartment bipolar membrane cell, the base product stream comprising sodium hydroxide. The salt product stream is contacted with a crystallizer, thereby forming a crystallizer stream. The crystallizer stream is contacted with a filtration system, thereby forming a solid product stream comprising iminodiacetic acid and a recirculation stream. The recirculation stream is combined with the feed salt stream prior to introduction into the salt compartment of the two-compartment electrodialysis bipolar membrane cell. At least a portion of the feed salt stream comprising DSIDA is prepared by reacting the base product stream comprising sodium hydroxide with diethanolamine (DEA) in the presence of a catalyst

The present disclosure is further directed to a process for preparing iminodiacetic acid wherein the process comprises introducing a feed salt stream comprising disodium iminodiacetic acid (DSIDA) into a salt compartment of a two-compartment electrodialysis bipolar membrane cell comprising the salt compartment and a base compartment. A salt product stream is recovered from the salt compartment of the two-compartment bipolar membrane cell, the salt product stream comprising iminodiacetic acid (IDA) and monosodium iminodiacetic acid (MSIDA). A base product stream is recovered from the base compartment of the two-compartment bipolar membrane cell, the base product stream comprising sodium hydroxide. The salt product stream is contacted with a crystallizer, thereby forming a crystallizer stream. The crystallizer stream is contacted with a filtration system, thereby forming a solid product stream comprising IDA and a recirculation stream. The recirculation stream is combined with the feed salt stream prior to introduction into the salt compartment of the two-compartment electrodialysis bipolar membrane cell. The process further comprises phosphonomethylating the IDA in the solid product stream.

The present disclosure is also directed to a process for preparing iminodiacetic acid wherein the process comprises introducing a feed salt stream comprising disodium iminodiacetic acid (DSIDA) into a salt compartment of a two-compartment electrodialysis bipolar membrane cell comprising the salt compartment and a base compartment. A salt product stream is recovered from the salt compartment of the two-compartment bipolar membrane cell comprising the amino acid, the salt product stream comprising iminodiacetic acid (IDA) and monosodium iminodicacetic acid (MSIDA). A base product stream is recovered from the base compartment of the two-compartment bipolar membrane cell, the base product stream comprising sodium hydroxide. The salt product stream is contacted with a crystallizer, thereby forming a crystallizer stream. The crystallizer stream is contacted with a filtration system, thereby forming a solid product stream comprising the iminodiacetic acid and a recirculation stream. The recirculation stream is combined with the feed salt stream prior to introduction into the salt compartment of the two-compartment electrodialysis bipolar membrane cell. At least a portion of the feed salt stream comprising DSIDA is prepared by reacting the base product stream comprising sodium hydroxide with diethanolamine in the presence of a catalyst to form DSIDA. The process further comprises phosphonomethylating the iminodiacetic acid in the solid product stream.

The present disclosure is further directed to a process for preparing iminodiacetic acid, wherein the process comprises introducing a feed salt stream comprising disodium iminodiacetic acid (DSIDA) into a salt compartment of a two-compartment electrodialysis bipolar membrane cell comprising the salt compartment and a base compartment. A salt product stream is recovered from the salt compartment of the two-compartment bipolar membrane cell, the salt product stream comprising iminodiacetic acid (IDA) and monosodium iminodicacetic acid (MSIDA). A base product stream is recovered from the base compartment of the two-compartment bipolar membrane cell, the base product stream comprising sodium hydroxide. The salt product stream is contacted with a crystallizer, thereby forming a crystallizer stream. The base product stream is essentially chloride (Cl⁻) free and at least a portion of the feed salt stream comprising DSIDA is prepared by reacting the base product stream comprising sodium hydroxide with diethanolamine (DEA) in the presence of a catalyst.

Other objects and features will be in part apparent and in part pointed out hereinafter.

### BRIEF DESCRIPTION OF THE DRAWINGS

FIG. 1a shows an example configuration of a two-compartment bipolar membrane electrodialysis cell and the flow of the respective ions when subjected to an electric potential between a cathode and anode.

FIG. 1b shows an example configuration of a two-compartment bipolar membrane electrodialysis cell with a cationic exchange membrane (CEM) as the end membranes.

FIG. 2 shows a 2-compartment BME process in the context of an overall DSIDA conversion and recirculation process.

FIG. 3 shows the current efficiency and power usage of a 2-compartment bipolar membrane electrodialysis process at various pH values.

FIG. 4 shows the speciation of IDA, MSIDA, and DSIDA during the process of Example 3.

FIG. 5 shows the conductivity and pH of the salt compartment of the 2-compartment bipolar membrane electrodialysis apparatus over time for the initial (no recirculation) cycle of the system in Example 4.

FIG. 6 shows the conductivity and pH of the salt compartment of the 2-compartment bipolar membrane electrodialysis apparatus over time during the third recirculation cycle of the process of Example 5.

FIG. 7 demonstrates the conductivity of the base compartment of the 2-compartment bipolar membrane electrodialysis apparatus over time during the third recirculation cycle of the process of Example 5.

FIG. 8 shows the current and voltage of the 2-compartment BME system during the third recirculation cycle of Example 6.

FIG. 9 shows the current and voltage of a comparative 3-compartment BME system of Example 6.

### DETAILED DESCRIPTION

Provided herein is a two-compartment bipolar membrane electrodialysis apparatus and processes for producing an amino acid using the two-compartment bipolar membrane apparatus, wherein the feed stream comprises a salt of the amino acid. As described herein, the feed stream to the salt compartment of the two-compartment bipolar membrane apparatus may be a starting amino acid salt feed stream, a recycle salt stream recovered from the process of the present disclosure, or a combination thereof.

As detailed elsewhere herein, the electrodialysis processes of the present disclosure, are suitable for integration with processes for preparing amino acid salts. Certain catalysts used for preparing a salt of an amino acid (e.g., DSIDA) may be sensitive to the presence of chloride. For example, when chloride is present in concentrations of more than 200 ppm, a detrimental impact on the catalyst for preparing the salt of an amino acid (e.g., DSIDA) may be observed, including deactivation and/or poisoning of the catalyst by such concentrations of chloride. The base product stream of the present processes exhibits levels of chloride content that avoid catalyst deactivation and/or poisoning issues. In fact, typically in accordance with the present disclosure the base product stream is essentially chloride-free, and in certain embodiments is chloride-free.

Accordingly, two-compartment bipolar membrane electrodialysis processes of the present disclosure result in a base product stream of the two-compartment bipolar membrane that is substantially chloride free and can be utilized in the process for preparing the salt of an amino acid without fear of significant catalyst deactivation or poisoning. Described herein are processes that allow for a substantially chloride free base product stream, and improved overall process efficiency and commercially acceptable yields of the desired amino acid.

The present disclosure also relates to a two-compartment bipolar membrane electrodialysis process for preparing an amino acid (e.g., iminodiacetic acid—i.e., IDA) from a salt of the amino acid (e.g., disodium iminodiacetic acid—i.e., DSIDA) wherein the base product is substantially chloride free. For example, the present disclosure does not result in the formation of chloride when preparing IDA from DSIDA. Although sodium salts are discussed herein, in certain embodiments the present disclosure relates to preparing an amino acid from a salt of the amino acid, wherein the salt comprises a cation other than sodium. Suitable salt cations may be selected, for example, from the group consisting of potassium, lithium, ammonium, calcium, and magnesium.

Further, as detailed below, the present disclosure also relates to an electrodialysis process utilizing a two-compartment bipolar membrane apparatus for preparing an amino acid from a salt of the amino acid. In accordance with such embodiments, the two-compartment bipolar membrane converts the amino acid salt in a feed salt stream to the desired amino acid, the salt product stream is treated with a crystallizer and then subjected to filtration to form a solid product stream comprising the amino acid and a recirculation stream. At least a portion of the recirculation stream is combined with the feed salt stream prior to introduction into the two-compartment electrodialysis bipolar membrane cell. The solid product stream comprising the amino acid may be further processed or purified. In certain embodiments, the process further comprises phosphonomethylating the amino acid in the solid product stream.

The present disclosure is also directed to a process for preparing an amino acid (e.g., IDA) from a salt of the amino acid (e.g., disodium iminodiacetic acid, i.e., DSIDA) utilized a two-compartment bipolar membrane apparatus, wherein at least a portion of the feed salt stream comprising a salt of the amino acid is prepared by reacting the base product stream of the two-compartment bipolar membrane apparatus with an ethanolamine in the presence of a catalyst to form a salt of the amino acid.

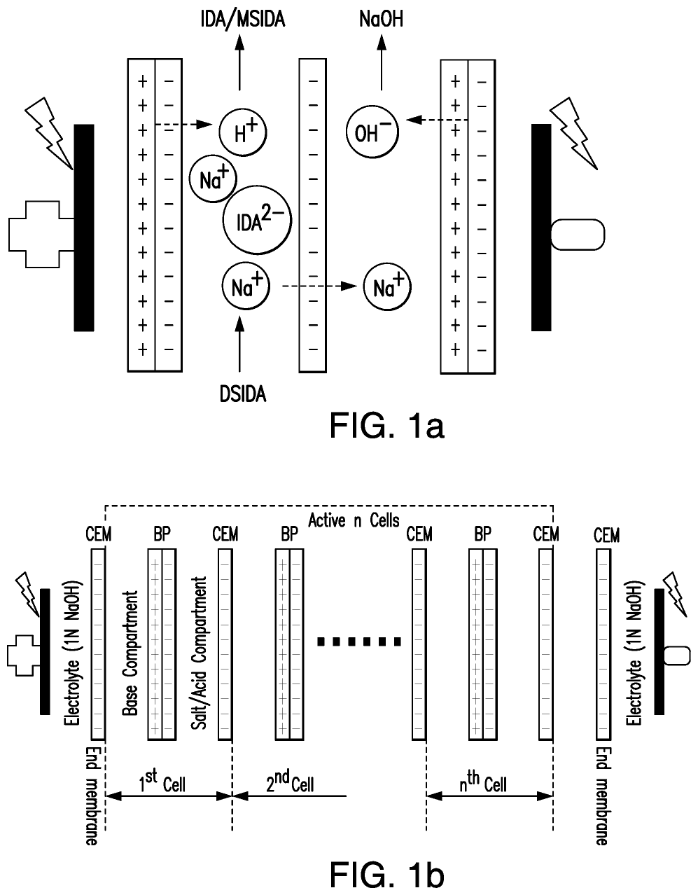

In various embodiments of the present disclosure, the two-compartment bipolar membrane apparatus comprises one or more repeating units (i.e., “membrane units”) comprising a bipolar membrane (BPM) and a cation exchange membrane (CEM). The one or more repeating membrane units may be, for example, the following configurations: [BPM-CEM]ₙ, [BPM¹-CEM-BPM²]ₙ, or [CEM¹-BPM-CEM²]ₙ wherein n is the number of repeating units. For example, where the membrane cell comprises one or more repeating membrane unit(s), an anode, and a cathode, generally the bipolar membrane apparatus is characterized by the following configuration: Anode-{[CEM¹-BPM-CEM²]ₙ}-Cathode or Anode-{[BPM¹-CEM-BPM²]ₙ}-Cathode. Non-limiting examples of this can be seen in FIGS 1a and 1b. For example, the bipolar membrane apparatus may comprise any of the above configurations of repeating membrane units wherein n can be any whole number. For example, n may be 10, 20, 30, 40, 50, 60, 70, 80, 90, 100, 110, 120, 130, 140, 150, 170, 190, 210, 230, 250, 270, 290, or 300. In certain preferred embodiments, n is 7. In other preferred embodiments, n is a whole number from about 1 to about 300 or from about 1 to about 200.

Generally, along with the membrane cell, anode, and cathode, the two-compartment bipolar membrane apparatus of the present disclosure may include one or more terminal or end membranes positioned between the one or more repeating membrane units and the anode and/or between the one or more repeating membrane units and the cathode. The terminal or end membrane(s) may be a CEM or BPM. In certain embodiments, the terminal or end membrane(s) is a CEM.

In certain embodiments, the two-compartment membrane cell, comprising one or more repeating membrane units, begins with a bipolar membrane and terminates with a bipolar membrane. For example, the membrane cell may comprise one or more repeating [BPM-CEM] membrane units and be of the following configuration: Anode-{[BPM¹-CEM-]ₙBPM²}-Cathode, wherein n can be any whole number from 1 to 200. For example, the membrane cell may be of the configuration: BPM¹-CEM-BPM² as shown in FIG 1a. In certain other embodiments, the membrane cell may comprise one or more repeating [CEM-BPM] membrane units and be of the following configuration: Anode-{[CEM¹-BPM-]ₙCEM²}-Cathode, wherein n can be any whole number from 1 to 200. For example, as shown in FIG. 1b.

By utilizing one of the above mentioned configurations, the two-compartment membrane cell forms one or more distinct salt and base compartments. For example, in the embodiment of FIG. 1a, the base compartment is bounded by a first bipolar membrane and a cationic exchange membrane and the salt compartment is bounded by the cationic exchange membrane of the base compartment and a second bipolar membrane. Embodiments wherein the membrane cell comprises one or more repeating membrane units may be configured such that the one or more repeating membrane units terminates on each end with a bipolar membrane to allow for water splitting to occur at a location immediately adjacent to each base compartment. Embodiments wherein the membrane cell comprises one or more repeating membrane units and is configured such that the one or more repeating membrane units terminates on each end with a cation exchange membrane allows for the introduction of a basic solution adjacent to the cathode and anode. This configuration is shown, for example, in FIG. 1b.

In the bipolar membrane electrodialysis process of the present disclosure, the two-compartment bipolar membrane cell comprising one or more repeating membrane unit(s) is located between a cathode at one end and an anode at the other end. When an electric potential is applied water will split into H⁺ and OH⁻ ions. The electric potential will thereby induce the H⁺ ions to permeate through the cation-exchange side of bipolar exchange membrane towards the salt compartment. The electric potential also induces flow of OH⁻ ions through the anion-exchange side of the bipolar exchange membrane towards the base compartment. The salt cation will likewise migrate through the cation exchange membrane towards the base compartment.

From this process, the anions from the salt of the amino acid and the protons combine in the salt compartment to form the amino acid. During this process, the anions from the salt of the amino acid may also combine with residual salt cations to form a salt of the amino acid. At the same time, the hydroxide ions combine in the base compartment with the salt cations to form a base.

For example, in a process wherein the feed salt comprises DSIDA, the H⁺ ions will combine with the IDA anions and gradually convert DSIDA (a salt of the amino acid) into monosodium iminodiacetic acid, i.e., MSIDA, (a salt of the amino acid) and IDA (the amino acid). The sodium cations from salt compartment migrate through the cation exchange membrane and combine with the OH⁻ ions to form NaOH in the base compartment. FIG. 1a shows an example of the configuration of a membrane cell comprising a single BPM¹-CEM-BPM² membrane unit and the flow of the respective ions when subjected to an electric potential between a cathode and anode.

### Amino Acid

Although reference is made herein to the amino acid iminodiacetic acid (IDA) and the amino acid salt disodium iminodiacetic acid (DSIDA) and/or monosodium iminodiacetic acid (MSIDA), it understood that the apparatuses and processes described herein are applicable to numerous other amino acids and their salts.

The amino acid IDA is an essential component in the production of glyphosate (i.e. N-(phosphonomethyl)glycine). However, conventional methods for the production of IDA typically result in the formation of a sodium chloride salt as a waste product. The further processing of this waste product for proper disposal requires considerable cost and effort. Therefore, it is desirable to produce IDA through a process that does not result in formation of a sodium chloride salt waste product.

In various embodiments of the present disclosure, the amino acid has the following structure:

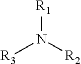

wherein R₁ is selected from the group consisting of CH₂C(O)OH, CH₂P(O)(OH)₂, and hydrogen; R₂ is selected from the group consisting of CH₂C(O)OH, CH₂P(O)(OH)₂, and hydrogen; and R₃ is selected from the group consisting of CH₂C(O)OH, CH₂P(O)(OH)₂, and hydrogen. In a preferred embodiment, R₁, R₂, and R₃ are independently selected from the group consisting of CH₂C(O)OH, CH₂P(O)(OH)₂, and hydrogen.

In further embodiments the amino acid is selected from the group consisting of iminodiacetic acid (including disodium iminodiacetic acid and monosodium iminodiacetic acid), N-(phosphonomethyl)iminodiacetic acid, glycine, and N-(phosphonomethyl)glycine.

In further embodiments the amino acid is selected from the group consisting of alanine, serine, threonine, cysteine, valine, leucine, isoleucine, methionine, proline, phenylalanine, tyrosine, tryptophan, asparitic acid, glutamic acid, asparagine, glutamine, histidine, lysine, and arginine, and salts thereof. Suitable salt cations may be selected, for example, from the group consisting of potassium, lithium, ammonium, calcium, and magnesium.

In certain preferred embodiments, the amino acid is iminodiacetic acid.

### Two-Compartment Bipolar Membrane Apparatus

### Salt Compartment

In the process of the present disclosure, a feed salt stream comprising a salt of the amino acid is introduced into the salt compartment of the two-compartment bipolar membrane apparatus. The electric potential of the electrodialysis process induces formation of amino acid anions from the salt of the amino acid in the salt compartment. Likewise, the electric potential induces formation of amino acid cations from the salt of the amino acid in the salt compartment and transport of the amino acid cations through the cationic exchange membrane and into the base compartment. An example of this transport of cations and anions from the inlet salt stream comprising a salt of an amino acid can be seen in FIG. 1a. In one embodiment, the stream exiting the salt compartment is substantially depleted in content of the salt of the amino acid.

In certain embodiments, the concentration of salt of the amino acid in the feed salt stream may be at least about 5 wt %, at least about 10 wt %, at least about 20 wt %, at least about 30 wt %, %, at least about 35 wt %, %, at least about 40 wt %, %, at least about 45 wt %, or at least about 50 wt %. For example, the concentration of salt of the amino acid in the feed salt stream may be from about 5 wt % to about 60 wt %, from about 10 wt % to about 50 wt %, from about 15 wt % to about 50 wt %, from about 20 wt % to about 50 wt %, from about 25 wt % to about 50 wt %, from about 30 wt % to about 50 wt %, from about 35 wt % to about 50 wt %, from about 40 wt % to about 50 wt %, or from about 40 wt % to about 45 wt %.

The contents of the salt compartment after introduction of the feed salt stream, in addition to the salt of the amino acid, may comprise amino acid anions, amino acid cations, ions from the water-splitting operation of the bipolar membrane, water, or any combination thereof.

In certain embodiments, the concentration of salt of the amino acid in the salt compartment may be at least about 1 wt %, at least about 5 wt %, at least about 10 wt %, at least about 15 wt %, at least about 20 wt %, at least about 25 wt %, at least about 30 wt %, at least about 35 wt %, at least about 40 wt %, or at least about 45 wt %. For example, the concentration of salt of the amino acid in the salt compartment may be from about 5 w t% to about 45 wt %, from about 10 wt % to about 35 wt %, from about 10 wt % to about 30 wt %, from about 15 wt % to about 30 wt %, or from about 20 wt % to about 30 wt %.

In certain embodiments, the conductivity of the salt stream introduced into the salt compartment is at least about 10 mS/cm, at least about 20 mS/cm, at least about 25 mS/cm, at least about 50 mS/cm, at least about 100 mS/cm, at least about 150 mS/cm, at least about 200 mS/cm, or at least about 250 mS/cm. In another embodiment, the conductivity of the salt stream introduced into the salt compartment is between about 10 and about 250 mS/cm, between about 20 and about 200 mS/cm, between 25 and about 200 mS/cm, between about 50 and about 200 mS/cm, between about 100 and about 200 mS/cm, or between about 150 and about 200 mS/cm.

In another embodiment, the conductivity of the content of the salt compartment is less than about 200 mS/cm, less than about 100 mS/cm, less than about 75 mS/cm, or less than about 50 mS/cm. For example, in certain embodiments, the conductivity of the content of the salt compartment is from about 200 mS/cm to about 0 mS/cm, from about 100 mS to about 0 mS/cm, from about 75 to about 0 mS/cm, or from about 50 mS/cm to about 0 mS/cm.

As described in further detail below, wherein the process comprises an initial cycle with no recirculation (i.e. wherein the feed stream to the salt compartment of the two-compartment bipolar exchange membrane is comprised primarily of the feed stream), the conductivity of the salt compartment during this non-recirculation cycle may be at least about 10 mS/cm, at least about 20 mS/cm, at least about 30 mS/cm, at least about 40 mS/cm, or at least about 50 mS/cm. For example, between about 10 and about 200 mS/cm, between about 10 and about 150 mS/cm, between 10 and about 100 mS/cm, between about 15 and about 100 mS/cm, between about 20 and about 100 mS/cm, between about 25 and about 100 mS/cm, between about 30 and about 100 mS/cm, between about 30 and about 90 mS/cm, between about 30 and about 80 mS/cm, between about 35 and about 80 mS/cm, between about 40 and about 80 mS/cm, between about 40 and about 70 mS/cm, or between about 40 and about 60 mS/cm.

Additionally, wherein the process comprises a recirculation stream, the conductivity of the salt compartment during the third recirculation cycle or greater may be between 10 and about 100 mS/cm, between about 10 and about 90 mS/cm, between about 10 and about 80 mS/cm, between about 10 and about 70 mS/cm, between about 10 and about 60 mS/cm, between about 20 and about 60 mS/cm, between about 30 and about 60 mS/cm, between about 35 and about 55 mS/cm, or between about 40 and about 50 mS/cm.

In certain embodiments, the process further comprises recovering a salt product stream comprising the amino acid from the salt compartment. For example, in certain embodiments, the amino acid constitutes at least about 2 wt %, at least about 4 wt %, at least about 6 wt %, at least about 8 wt %, at least about 10 wt %, at least about 12 wt %, at least about 14 wt %, at least about 16 wt %, at least about 18 wt %, or at least about 20 wt % of the salt product stream. In another embodiment, the amino acid constitutes from about 2 to about 20 wt %, from about 4 wt % to about 18 wt %, from about 6 wt % to about 16 wt %, from about 6 wt % to about 14 wt %, from about 8 wt % to about 14 wt %, or from about 8 wt % to about 12 wt % of the salt product stream.

In certain embodiments, the salt product stream further comprises a salt of the amino acid (e.g., MSIDA) different than the salt of the amino acid introduced in the feed stream (DSIDA). For example, when the salt of the amino acid introduced in the feed stream is DSIDA, the salt product stream may comprise at least about 5 wt. %, at least about 10 wt. %, at least about 15 wt. %, or at least about 20 wt.% MSIDA. In one embodiment, the salt product stream may comprise less than about 30 wt. %, less than about 25 wt.%, less than about 20 wt. %, less than about 15 wt. %, less than about 10 wt. %, or less than about 5 wt. % MSIDA.

In certain embodiments, the amino acid content of the salt product stream represents a yield based on the amino acid salt introduced into the salt compartment

$$( e . g . , \frac{moles iminodiacetic acid recovered from salt compartment}{moles iminodiacetic {acid}^{- 2} in DSIDA feed} \times 100 ) .$$

For example, the yield may be at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. For example, in certain embodiments, at least about 80% of the salt of the amino acid introduced into the salt compartment is converted to the amino acid recovered in the salt product stream. In a preferred embodiment, the target yield of amino acid is at least about 80%, at least about 85%, at least about 90%, or at least about 95%.

The yield based on the amino acid salt introduced into the salt compartment

$$( e . g . , \frac{moles iminodiacetic acid recovered from salt compartment}{moles iminodiacetic {acid}^{- 2} in DSIDA feed} \times 100 )$$

may be evaluated at the conclusion of a certain cycle. As detailed elsewhere herein, a “cycle” begins at the point where the feed stream is introduced into the salt compartment. This feed stream can be introduced as the lone feed stream to the salt compartment during initial operation (e.g., during an initial non-recirculation cycle) and is typically combined with a recirculation stream in later cycles.

A cycle in which the feed stream is introduced as the primary or lone feed stream to the salt compartment of the of the two-compartment bipolar exchange membrane is characterized herein as a “non-recirculation cycle,” “cycle with without recirculation,” etc. A cycle in which the feed stream is combined with a recirculation stream and introduced into the salt compartment of the of the two-compartment bipolar exchange membrane is characterized herein as a “recirculation cycle.” As discussed in greater detail below, in certain embodiments, the process comprises a continuous recirculation stream or one or more continuous recirculation cycles. In those embodiments, the cycle may be referenced based on the number of recirculation cycles. For example, “first recirculation cycle,” “second recirculation cycle,” “recirculation cycle 1,” recirculation cycle X,” etc., wherein x is a positive integer.

At the conclusion of the first recirculation cycle, the yield based on the amino acid salt introduced into the salt compartment

$$( e . g . , \frac{moles iminodiacetic acid recovered from salt compartment}{moles iminodiacetic {acid}^{- 2} in DSIDA feed} \times 100 )$$

may be at least about 20%, at least about 25%, at least about 30%, or at least about 35%. In certain embodiments, after conclusion of the third recirculation cycle the yield may be at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In these and other embodiments, after conclusion of the twentieth recirculation cycle the yield may be at least about 80%, at least about 85%, at least about 90%, at least about 91%, at least about 92%, at least about 93%, at least about 94%, at least about 95%, at least about 96%, at least about 97%, at least about 98%, or at least about 99%. In certain embodiments, the process of the present disclosure may demonstrate a yield of at least the values set forth in the below table for a given cycle, wherein the yield is measured at the conclusion of the noted cycle.

| Cycle | IDA yield |
|---|---|
| Non-Recirculation Cycle | 23% |
| Recirculation Cycle 1 | 37% |
| Recirculation Cycle 2 | 47% |
| Recirculation Cycle 3 | 54% |
| Recirculation Cycle 20 | 86% |
| Recirculation Cycle 100 | 97% |
| Recirculation Cycle 200 | 98% |

In certain embodiments, the salt product stream comprising less than about 20 wt %, less than about 15 wt %, less than about 10 wt %, less than about 5 wt %, less than about 4 wt %, less than about 3 wt %, less than about 2 wt %, less than about 1 wt %, or less than about 0.5 wt % of the salt of the amino acid.

In certain embodiments, during operation, the salt compartment has a pH from about 2 to about 13. For example, during operation, the salt compartment may have a pH from about 2 to about 12, from about 2 to about 11, from about 3 to about 11, or from about 3 to about 10.

In one embodiment, the salt product stream of the two-compartment electrodialysis bipolar membrane of the present disclosure has a chloride content of less than about 200 ppm, less than about 100 ppm, less than about 50 ppm, less than about 25 ppm, less than about 20 ppm, less than about 10 ppm, less than about 5 ppm, or less than about 1 ppm.

### Base Compartment

As set forth above, the electric potential of the electrodialysis process induces flow of hydroxide ions toward the anode and formation of amino acid cations from the salt of the amino acid in the salt compartment, wherein the amino acid cations pass through the cationic exchange membrane and into the base compartment of the two-compartment bipolar membrane apparatus. The cations from the salt of the amino acid and hydroxide ions from the water-splitting operation of the bipolar membrane combine in the base compartment to form a base. This can be seen, for example, in FIG. 1a.

The contents of the base compartment may comprise cations of the salt of the amino acid, ions from the water-splitting operation of the bipolar membrane, water, or any combination thereof.

In certain embodiments, the conductivity of the content of the base compartment is at least about 10 mS/cm, at least about 20 mS/cm, at least about 50 mS/cm, at least about 100 mS/cm, at least about 150 mS/cm, at least about 200 mS/cm, at least about 250 mS/cm, at least about 300 mS/cm, at least about 350 mS/cm, or at least about 400 mS/cm. For example, in certain embodiments, the conductivity of the content of the base compartment is from about 10 mS/cm to about 500 mS/cm from about 10 mS/cm to about 400 mS/cm, from about 50 mS/cm to about 400 mS/cm, from about 50 mS/cm to about 350 mS/cm, from about 100 mS/cm to about 350 mS/cm, from about 150 mS/cm to about 350 mS/cm, from about 200 mS/cm to about 350 mS/cm, from about 200 mS/cm to about 300 mS/cm, or from about 200 mS/cm to about 250 mS/cm.

In yet a further embodiment, the process further comprises recovering a base product stream from the base compartment. In certain embodiments, the base content of the base product stream represents a yield based on the cation of the amino acid salt (e.g., (moles NaOH recovered from base compartment)/(moles Na⁺ in DSIDA feed)×100) of at least about 90%, at least about 91%, at least about 92%, at least about 93%, at least about 94%, at least about 95%, at least about 96%, at least about 97%, at least about 98%, or at least about 99%.

In certain embodiments, the base product stream comprises at least about 5 wt. %, at least about 10 wt. %, at least about 15 wt. %, at least about 20 wt. %, at least about 25 wt. %, at least about 30 wt. %, at least about 35 wt. %, or at least about 40 wt. % of a base. For example, in a process wherein the feed salt stream comprises DSIDA and/or MSIDA in a total combined concentration of at least about 20 wt. %, the base product stream comprises at least about 5 wt. %, at least about 10 wt. %, at least about 15 wt. %, at least about 20 wt. %, at least about 25 wt. %, at least about 30 wt. %, at least about 35 wt. %, or at least about 40 wt. % NaOH.

In one embodiment, the base product stream of the two-compartment electrodialysis bipolar membrane of the present disclosure has a chloride content of less than about 200 ppm, less than about 100 ppm, less than about 50 ppm, less than about 25 ppm, less than about 20 ppm, less than about 10 ppm, less than about 5 ppm, or less than about 1 ppm.

### Membranes

Suitable cationic exchange membranes are commercially available from manufacturers such as Suez Water Technologies, Astom (e.g., NEOSEPTA), Fumatech, Allied Corporation, Tokuyama Soda, and WSI Technologies.

Suitable bipolar membranes are commercially available from manufacturers such as Suez Water Technologies, Astom (e.g., NEOSEPTA), Fumatech, Allied Corporation, Tokuyama Soda, Eurodia Industrie SA, and WSI Technologies.

### Power Usage and Efficiency

In certain embodiments, applying an electric potential between the cathode and the anode of the two-compartment electrodialysis bipolar membrane comprises application of at least about 1 A (amps), at least about 5 A, at least about 6 A, at least about 7 A, at least about 8 A, at least about 9 A, at least about 10 A, at least about 11 A, at least about 12 A, at least about 13 A, at least about 14 A, or at least about 15 A. For example, in one embodiment, applying an electric potential between the cathode and the anode of the two-compartment electrodialysis bipolar membrane comprises application of about 14 A.

In another embodiment, applying an electric potential between the cathode and the anode of the two-compartment electrodialysis bipolar membrane comprises application of at least about 5 V (volts), at least about 10 V, at least about 15 V, at least about 20 V, or at least about 25 V. In one embodiment, applying an electric potential between the cathode and the anode of the two-compartment electrodialysis bipolar membrane comprises application of less than about 30 V, less than about 25 V, or less than about 20 V.

In certain embodiments, the current efficiency based on the transport of the cation of the salt of the amino acid to the base compartment of the two-compartment electrodialysis bipolar membrane is determined. The current efficiency can be calculated using the following formula:

$$\frac{Moles of {Na}^{+} converted}{Moles of electrons provided}$$

wherein the moles of electrons provided is determined by the formula:

$$( total number of repeating membrane units ) \times \frac{I \cdot t}{F} .$$

I is the current intensity reported in units of amps or coulombs, F is the faraday constant (96,485 C mol⁻¹), and t represents time.

For example, the current efficiency is at least about 75%, at least about 80%, at least about 85%, at least about 87%, at least about 89%, at least about 91%, at least about 93%, at least about 95%, at least about 96%, at least about 97%, at least about 98%, or at least about 99%. For example, in certain embodiments, the current efficiency based on the transport of the cation of the salt of the amino acid to the base compartment is from about 80% to about 99%, from about 81% to about 99%, from about 82% to about 99%, from about 83% to about 99%, from about 84% to about 99%, from about 85% to about 99%, from about 86% to about 99%, from about 87% to about 99%, from about 88% to about 99%, from about 89% to about 99%, from about 90% to about 99%, or from about 95% to about 99%.

In certain embodiments, the power usage is less than about 5 kW/hr, less than about 4 kW/hr, less than about 3 kW/hr, less than about 2 kW/hr, less than about 1 kW/hr, less than about 0.75 kW/hr, less than about 0.7 kW/hr, less than about 0.65 kW/hr, or less than about 0.6 kW/hr.

In certain embodiments, the specific power usage is less than about 3,000 kWhr/Ton base, less than about 2,900 kWhr/Ton base, less than about 2,800 kWhr/Ton base, less than about 2,700 kWhr/Ton base, less than about 2,6000 kWhr/Ton base, less than about 2,500 kWhr/Ton base, less than about 2,400 kWhr/Ton base, less than about 2,300 kWhr/Ton base, less than about 2,200 kWhr/Ton base, less than about 2,100 kWhr/Ton base, less than about 2,050 kWhr/Ton base, less than about 2,000 kWhr/Ton base, less than about 1,950 kWhr/Ton base, less than about 1,900 kWhr/Ton base, less than about 1,850 kWhr/Ton base, less than about 1,800 kWhr/Ton base, less than about 1,750 kWhr/Ton base, less than about 1,500 kWhr/Ton base, less than about 1,250 kWhr/Ton base, or less than about 1,000 kWhr/Ton base.

### Salt of the Amino Acid Production

In certain embodiments, the base product of the two-compartment bipolar membrane apparatus may be utilized in forming the salt of the amino acid introduced to the two-compartment bipolar membrane apparatus. For example, the salt of the amino acid introduced to the two-compartment bipolar membrane apparatus may be formed using at least a portion of the base product stream of the two-compartment bipolar membrane apparatus by any processes known in the art.

In certain embodiment, at least a portion of the base product stream of the two-compartment bipolar membrane apparatus may be further processed to prepare a concentrated base product stream. For example, the base product stream may be concentrated by known methods including, for example, evaporation. In one embodiment, the base product stream is subjected to evaporation under vacuum conditions and under a controlled temperature (e.g., about 45° C. or less).

In certain embodiment, the concentrated base product stream may comprise at least about 10 wt. %, at least about 15 wt. %, at least about 20 wt. %, at least about 25 wt. %, at least about 30 wt. %, at least about 35 wt. %, at least about 40 wt. %, at least about 45 wt. %, or at least about 50 wt. % of a base. For example, the concentrated base product stream may comprise from about 15 wt. % to about 50 wt. %, from about 20 wt. % to about 50 wt. %, from about 25 wt. % to about 50 wt. %, from about 30 wt. % to about 50 wt. %, from about 35 wt. % to about 50 wt. %, from about 35 wt. % to about 45 wt. %, or from about 35 wt. % to about 40 wt. % of a base.

Generally, the base utilized in the present processes is an alkali metal salt. In one embodiment, the base is a strong base. “Strong base” as used herein means a basic compound capable of deprotonating a weak acid in an acid-base reaction. For example, the strong base may be selected from the group consisting of hydroxides, alkoxides, and ammonia. In certain embodiments, the strong base may be, for example, sodium hydroxide, potassium hydroxide, barium hydroxide, cesium hydroxide, strontium hydroxide, lithium hydroxide, or rubidium hydroxide.

In certain embodiments, the base or strong base is sodium hydroxide (NaOH) or potassium hydroxide (KOH). In various embodiments, the base or strong base is sodium hydroxide.

In one embodiment, at least a portion of the feed salt stream comprising a salt of the amino acid is prepared by reacting at least a portion of the base product stream or concentrated base product stream with an ethanolamine in the presence of a catalyst to form a salt of the amino acid. For example, in one embodiment, the salt of the amino acid is DSIDA, and at least a portion of the DSIDA is formed by catalytic oxidation of diethanolamine in the presence of at least a portion of the base product stream or concentrated base product stream. In certain embodiments, the ethanolamine is diethanolamine. The catalyst used in this process may be any catalyst useful for such a process. Generally, the catalyst of this process is subject to poisoning or deactivation in the presence of chloride.

In another embodiment, the process for preparing the salt of the amino acid comprises dehydrogenation of the ethanolamine For example, dehydrogenation of diethanolamine.

### Processing and Recycle of the Salt Product Stream

The two-compartment bipolar membrane apparatus of the present disclosure allows for a substantially chloride free process. That is, the feed stream, base product stream, and salt product stream may all have a chloride content of less than about 200 ppm, less than about 100 ppm, less than about 50 ppm, less than about 25 ppm, less than about 20 ppm, less than about 10 ppm, less than about 5 ppm, or less than about 1 ppm. This is an important aspect of the present disclosure and allows for decreased operational cost by, for example, allowing recycle of the base product stream for use in formation of the salt of the amino acid without poisoning the catalyst used in said process for preparing the salt of the amino acid.

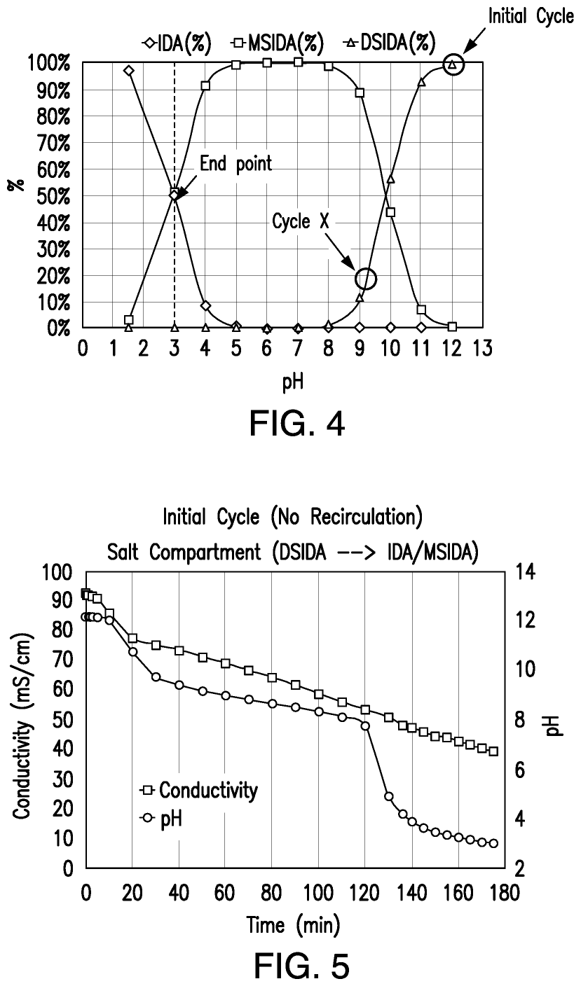

However, the two-compartment bipolar membrane apparatus of the present disclosure may convert less of the salt of the amino acid to the amino acid as compared to previously known three-compartment bipolar membrane apparatuses that introduce an exogenous acid (e.g., HCl) or otherwise operate with chloride present in the process. For example, when the salt of the amino acid is DSIDA, the two-compartment bipolar membrane apparatus has a limitation as to how much sodium can be converted. As sodium ions are transferred to the base compartment and protons formed from bipolar exchange membrane enter the salt compartment, the proton concentration in the salt compartment increases and the pH goes down. FIG. 3 graphically demonstrates the relationship between pH, sodium conversion, and current efficiency. For example, as the pH dropped from 3 to 2.5, the sodium conversion increased from 75% to 85%. However, during this period, the current efficiency dropped from 85% to 60%. It is generally desirable to operate at a current efficiency of at least about 85%. Notably, a single pass of the two-compartment bipolar membrane apparatus (i.e. a process comprising one non-recirculation cycle) can only achieve approximately 75% conversion of sodium without significant loss of current efficiency. FIG. 4 presents the speciation of IDA, MSIDA and DSIDA at different pH values.

Therefore, in order to operate the two-compartment bipolar membrane apparatus of the present disclosure in an economical manner for preparing an amino acid, one aspect of the present disclosure is directed to further processing of the salt product stream of the two-compartment bipolar membrane and recycling at least a portion of the processed salt product stream combined and combining the processed salt product stream with the feed stream.

Further processing of the salt product stream of the two-compartment bipolar membrane is accomplished by a variety of apparatuses including, for example, a crystallizer and/or a filtration system.

As set forth above, the salt product stream of the two-compartment bipolar membrane apparatus comprises the amino acid and in some embodiments further comprises one or more salts of the amino acid. In one aspect of the present disclosure, the salt product stream is fed to a crystallizer wherein at least a portion of the amino acid present therein is crystallized, thereby forming a crystallizer stream comprising an amino acid solid. The crystallizer, for example, may be selected from the group consisting of a dynamic crystallizer, a static crystallizer, a suspension crystallizer, a falling-film crystallizer, a tubular falling-film crystallizer, a melt crystallizer, or any combination thereof. In one embodiment, the crystallizer is selected from the group consisting of a batch cooling type crystallizer, a continuous cooling type crystallizer, a continuous evaporative type crystallizer, a batch evaporative type crystallizer, or any combination thereof.

In one embodiment, the salt product stream is cooled prior to being introduced into the crystallizer For example, the salt product stream may be cooled to less than about 30° C., less than about 25° C., less than about 20° C., less than about 15° C., or less than about 10° C. prior to being introduced into the crystallizer. In one embodiment, the salt product is cooled to between about 30° C. and about 10° C., between about 25° C. and about 10° C., between about 20° C. and about 10° C., or between about 20° C. and about 15° C. prior to being introduced into the crystallizer The solubility of the amino acid (e.g., IDA) decreases as the temperature decreases, thereby allowing for a greater recovery of an amino acid solid in the crystallizer. For example, IDA has a solubility of approximately 14 g IDA/100 g H₂O at a temperature of 60° C., but a solubility of less than 4 g IDA/100 g H₂O at a temperature of 10° C. The extent of the cooling of the salt product stream to ensure an economically viable process can therefore be determined by the solubility data of the subject amino acid to be recovered.

Another aspect of the further processing of the salt product stream involves contacting the crystallizer stream with a filtration system, thereby forming a solid product stream comprising the amino acid and a recirculation stream. The recirculation stream is then combined with the feed salt stream prior to introduction into the salt compartment of the two-compartment electrodialysis bipolar membrane cell.

The filtration system may comprise one or more filter, membrane, vacuum, centrifuge or any combination therefore. For example, in one embodiment, the filtration system comprises a House vacuum. In another embodiment, the filtration system comprises positive pressure driven filtration.

As used herein, each “cycle” is understood to begin at the point in time at which the feed stream is introduced into the salt compartment of the two-compartment bipolar membrane apparatus. The feed stream may be introduced as the only feed component including, for example, during initial operation (i.e. during a cycle without recirculation). The skilled artisan will understand that this is a definite point in time when the process is conducted in a batch manner However, when the process is operated as a continuous process, the exact point in time at which the new “cycle” begins may need to be determined by evaluating the profile of the stream introduced into the salt compartment of the two-compartment bipolar membrane apparatus or approximating the time at which the cycle begins through extrapolating previous measurements.

For example, the portion of the salt product stream that is recycled may be measured to determine its concentration of the salt of the amino acid. Knowing this concentration and the concentration of the salt of the amino acid of the feed stream, the skilled artisan can calculate the anticipated concentration of salt of the amino acid of the combined feed stream and recycle stream that is introduced into the salt compartment of the two-compartment bipolar membrane apparatus. The stream directed to the salt compartment of the two-compartment bipolar membrane apparatus can be measured to determine at what point in time the stream concentration transitions from a concentration that is consistent with only the presence of the feed stream, to a concentration that is consistent with the anticipated concentration of salt of the amino acid in a combined feed stream and recycle stream. This point in time would be considered the transition point to a new “cycle.” Measurement of the stream profile and approximation of new cycle time may be conducted by any known analytical and mathematical manner.

As noted above, typically at least a portion of the processed salt product stream is recycled (i.e. in a recirculation stream) and combined with the feed stream prior to introduction into the two-compartment bipolar membrane apparatus. Operation in this manner improves process economics. A certain number of recirculation cycles may be necessary to achieve the desired concentration of the amino acid in the solid product stream and/or to achieve the desired (high) yield of the amino acid.

In certain embodiments, the process of the present disclosure comprises at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18 at least about 19, at least about 20, at least about 25, at least about 30, or at least about 35 recirculation cycles. In one embodiment, the process of the present disclosure comprises 20 recirculation cycles.

In one embodiment, the solid product stream comprises at least about 10 wt. %, at least about 15 wt. %, at least about 20 wt. %, at least about 25 wt. %, at least about 30 wt. %, at least about 35 wt. %, at least about 40 wt. %, at least about 45 wt. %, at least about 50 wt. %, at least about 55 wt. %, at least about 60 wt. %, at least about 65 wt. %, at least about 70 wt. %, at least about 75 wt. %, at least about 80 wt. %, at least about 85 wt. %, or at least about 90 wt. % of the amino acid. For example, wherein the feed stream comprises the salt of the amino acid DSIDA and the process comprises at least about 20 recirculation cycles, the solid product stream comprises at least about 80 wt. % IDA.

### PMIDA and Glyphosate Production

The solid product stream resulting from contact of the crystallizer stream with a filtration system comprises the amino acid. This solid product stream is typically recovered in the form of a wetcake.

In one embodiment of the present disclosure, the process further comprises removing a slip stream comprising at least a portion of the recirculation stream prior to combination with the feed (salt) stream and removing water from the slip stream, thereby forming an additional solid product stream comprising the amino acid and one or more salt thereof.

The process further comprises phosphonomethylating the amino acid in the solid product stream or additional solid product stream to prepare N-(phosphonomethyl)iminodiacetic acid or a salt thereof (i.e., PMIDA). PMIDA can subsequently be converted to N-(phosphonomethyl)glycine or a salt thereof (i.e., glyphosate).

Processes for making PMIDA are known in the art and include those in which an alkali metal salt of iminodiacetic acid (IDA), a strong mineral acid, and a source of phosphorous acid are reacted. The disodium salt of IDA (DSIDA) is preferred. Suitable strong mineral acids include sulfuric, hydrobromic, hydroiodic and hydrochloric with hydrochloric generally preferred. In conventional methods, phosphorous acid can be added to the reaction medium or generated in situ by the hydrolysis of PCl₃. In such methods, PCl₃ is hydrolyzed to phosphorus acid in the DSIDA solution. HCl, which results from hydrolysis of phosphorous trichloride, acidifies the DSIDA to afford the hydrochloride salt and NaCl. Water vapor and a fraction of the HCl may be evolved from the reaction mixture and recovered in a hydrolysis reactor condenser. Optionally, a fraction of HCl driven from the reaction may be recycled to a subsequent batch. The IDA hydrochloride salt and NaCl are both substantially insoluble and form a slurry in an aqueous solution which is saturated with HCl. In a second step, the hydrolyzate slurry containing the strong acid salt of iminodiacetic acid, sodium chloride, hydrochloric acid and phosphorous acid is transferred from the hydrolyzer to a phosphonomethylation (PM) reactor. In the PM reactor, the slurry is combined with a source of formaldehyde to produce a PM reaction mixture containing PMIDA.

The present disclosure includes methods where P₄O₆ is utilized for the in situ preparation of phosphorous acid, thus avoiding the issues associated with PCl₃ detailed elsewhere herein. Such methods generally proceed as described above with the PCl₃ replaced by the P₄O₆.

The present disclosure is further directed to methods where P₄O₆ is utilized in a method for preparing PMIDA in a method that does not require the use of a strong mineral acid (e.g., hydrochloric acid). In such methods, P₄O₆ is hydrolyzed to form phosphorous acid, which is then reacted with formaldehyde and IDA to form PMIDA. In addition to avoiding the use of PCl₃ and the issues associated therewith, these methods therefore further avoid the issues associated with the use of hydrochloric, including the generation of chloride byproducts.

### EXAMPLES

### Example 1

A 2-compartment bipolar membrane electrodialysis process (BME) of the present disclosure was conducted with a feed comprising disodium iminodiacetate (DSIDA).

At startup, a 42 wt. % DSIDA feed stream was directed from a storage tank to the salt compartment of a two-compartment bipolar membrane electrodialysis process. The electrodialysis process was conducted at approximately 45° C. The bipolar exchange resin system of the process comprised an anode, first bipolar exchange membrane (BPM), cation exchange membrane (CEM), second bipolar exchange membrane, and a cathode. In this example, the configuration of the bipolar exchange system was Anode-[BPM₁-CEM-BPM₂]-Cathode. The DSIDA feed stream was introduced into the system into the salt compartment between the first bipolar exchange membrane (BPM₁) and cation exchange membrane. A stream comprising water was fed to the base compartment between the cation exchange membrane and the second bipolar exchange membrane (BPM₂).

An electric potential was applied between the cathode and anode, thereby inducing flow of protons toward the cathode and formation of amino acid anions from the salt of the amino acid in the salt compartment. The electric potential also induces flow of hydroxide ions toward the anode and formation of amino acid cations from the salt of the amino acid in the salt compartment, wherein the amino acid cations pass through the cationic exchange membrane and into the base compartment. The anions from the salt of the amino acid and the protons combined in the salt compartment to form the amino acid. The cations from the salt of the amino acid and the hydroxide ions combined in the base compartment to form a base. The resulting product stream of the salt compartment comprised iminodiacetic acid (IDA) and monosodium iminodiacetate (MSIDA), while the resulting product stream of the base compartment comprised NaOH.

FIG. 1a demonstrates the configuration of a membrane cell and the flow of the respective ions when subjected to an electric potential between a cathode and anode.

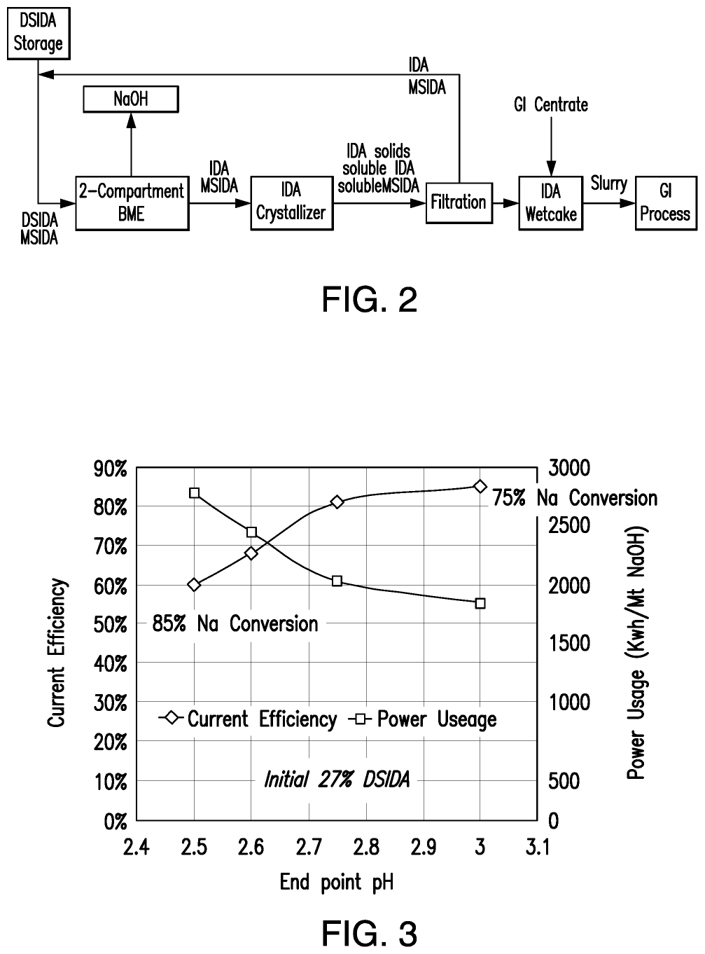

The product stream from the salt compartment was then directed to an iminodiacetic acid (IDA) crystallizer operating at 20° C. This resulted in a crystallizer stream comprising solid IDA, soluble IDA, and soluble MSIDA. The resulting crystallizer stream was then directed to a filtration system whereby the solid IDA was separated and dried, for use in downstream glyphosate production. The soluble IDA and MSIDA exiting the filtration system was recirculated and mixed with the feed stream for contact with the two-compartment bipolar membrane electrodialysis process. FIG. 2 shows the 2-compartment BME process in the context of this overall DSIDA conversion and recirculation process.

When the process had been started and the soluble IDA and MSIDA exiting the filtration system was continually recirculated as a source of the feed composition for the two-compartment bipolar membrane electrodialysis process, the following values were observed:

TABLE 1

| Combined Feed to 2- | | |
|---|---|---|
| Compartment BME | | |
| (i.e., DSIDA Feed + | Base Product | Salt Product |
| Recirculation Stream) | Stream | Stream |
| 8 wt. % DSIDA | 15 wt. % NaOH | 11 wt. % IDA |
| 17 wt. % MSIDA | | 15 wt. % MSIDA |

TABLE 2

| Salt Product Stream | Crystallizer Stream |
|---|---|
| 11 wt. % IDA | 8 wt. % IDA Solids |
| 15 wt. % MSIDA | 3 wt. % Soluble IDA |
| | 15 wt. % MSIDA |

TABLE 3

| Crystallizer Stream | Recirculation Stream |
|---|---|
| 8 wt. % IDA Solids | 3 wt. % IDA |
| 3 wt. % Soluble IDA | 16 wt. % MSIDA |
| 15 wt. % MSIDA | |

### Example 2

A further experiment was conducted to evaluate the conditions under which certain conversion rates of DSIDA could be achieved. The process was configured as set forth in Example 1.

The initial feed concentration of DSIDA to the salt compartment of the 2-compartment BME was approximately 27 wt. %. FIG. 3 represents the current efficiency and power usage of a 2-compartment bipolar membrane electrodialysis process at various pH values.

### Example 3

A further experiment was conducted, utilizing the configuration as set forth in Example 1, to evaluate the process at a certain number of cycles. The results are set forth in FIG. 4. “Initial cycle” corresponds to the step in the process wherein the feed to the 2-compartment BME was only the 42 wt. % DSIDA solution from the storage tank (i.e. a cycle without recirculation). Cycle 1 was the point in the process wherein the IDA/MSIDA recirculation stream from the filtration system had been recirculated a single time (i.e. recirculation cycle 1). Cycle X is the point in the process wherein the IDA/MSIDA stream from the filtration system has been recirculated x number of times, wherein x is an integer (i.e. the x numbered recirculation cycle).

FIG. 4 illustrates the speciation of IDA, MSIDA, and DSIDA during the process. As noted in the figure, the process started with a non-recirculation cycle wherein the iminodiacetic acid salt was substantially in the form of DSIDA (i.e. little or no IDA or MSIDA are present). At a certain point during the various recirculation cycles following the initial non-recirculation cycle, MSIDA began to represent a substantial portion of the stream, with DSIDA representing a smaller portion. The process was stopped at the final recirculation cycle, where little DSIDA was observed and near equal portions of MSIDA and IDA were present.

### Example 4

A further experiment was conducted utilizing the configuration of Example 1, in order to evaluate the conductivity and pH of the system during the initial non- recirculation cycle.

FIG. 5 demonstrates the conductivity and pH of the salt compartment of the 2-compartment bipolar membrane electrodialysis apparatus over time for the initial non- recirculation cycle of the system. The initial DSIDA feed concentration was approximately 27 wt. % and the exit NaOH stream produced from the 2-compartment BME had a NaOH concentration of approximately 15.4 wt. %. The current efficient was approximately 84% and the power usage of the 2-compartment BME was approximately 1840 Kwh/Mt NaOH.

### Example 5

A further experiment was conducted utilizing the configuration of Example 1, in order to evaluate the conductivity and pH of the system during the third recirculation cycle. The experiment was conducted by dividing the feed stream in half and introducing the divided feed stream to the salt stream in two batches.

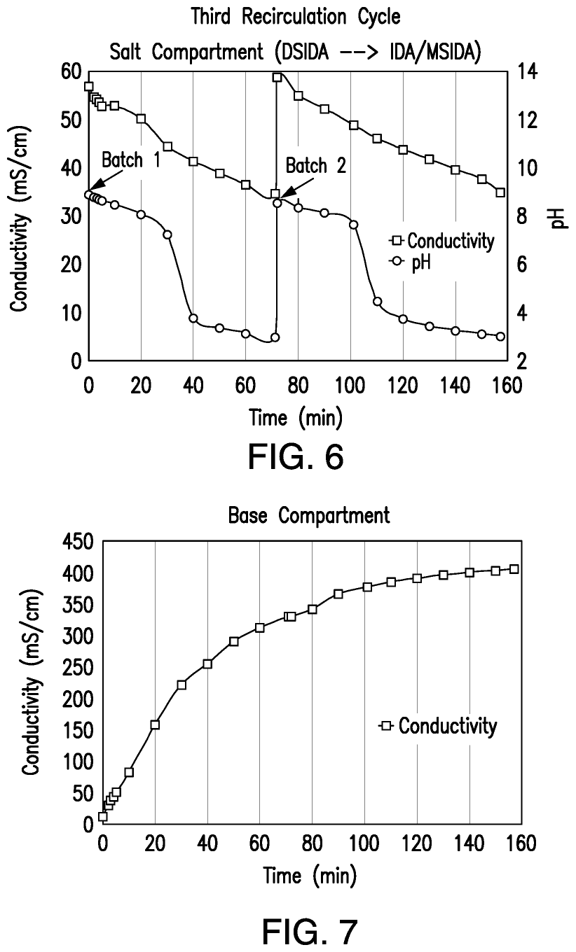

FIG. 6 demonstrates the conductivity and pH of the salt compartment of the 2-compartment bipolar membrane electrodialysis apparatus over time during the third recirculation cycle of the process. The base compartment was initially fed with a 0.1 M NaOH stream. After batch 1 (i.e. the first half of the feed stream) was introduced into the salt compartment, the salt compartment was discharged and a batch 2 (i.e. the second half of the feed stream) was introduced into the salt compartment. The initial feed to the 2-compartment BME for the third recirculation cycle was approximately 6 wt. % DSIDA and 16 wt. % MSIDA after combination with the recirculation stream of cycle 2. The current efficiency was approximately 83% and the power usage of the 2-compartment BME was maintained between 1,500 and 2,500 Kwb/Mt NaOH.

FIG. 7 demonstrates the conductivity of the base compartment of the 2-compartment bipolar membrane electrodialysis apparatus over time during the third recirculation cycle of the process.

### Example 6

Finally, an experiment was conducted to compare the 2-compartment BME process described above to a 3-compartment BME process.

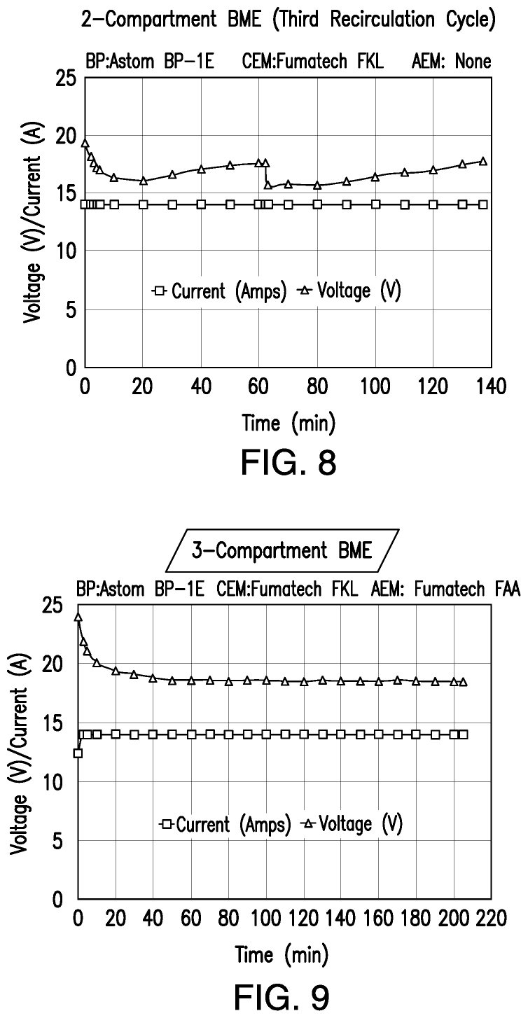

FIG. 8 reports the current and voltage of the 2-compartment BME system, utilizing the configuration set forth in Example 1, at the third recirculation cycle. The power usage was approximately 1,900 Kwb/Mt NaOH.

FIG. 9 reports the current and voltage of a comparative 3-compartment BME system. The power usage of the 3-compartment system was approximately 2,060 Kwb/Mt NaOH.

Further, Table 4 summarizes the NaOH production, current efficiency, and power usage of the 3-compartment BME process as compared to the 2-compartment BME process at the non-recirculation cycle and recirculation cycle 3.

TABLE 4

| | | 2-Compartment | 2-Compartment |
|---|---|---|---|
| | | BME (Non- | BME |
| | 3-Compartment | Recirculation | (Recirculation |
| | BME | Cycle) | Cycle 3) |
| NaOH Outlet | 15.3 wt. % | 15.4 wt. % | 15.5 wt. % |
| Current efficiency | 84% | 83% | 83% |
| Power usage | 2060 | 1840 | 1900 |
| (Kwh/MT NaOH) | | | |

The results of this experiment demonstrate that comparable NaOH production and current efficiency can be achieved by the present 2-compartment BME process. Additionally, the 2-compartment BME process achieves these results while utilizing lower amounts of power as compared to a 3-compartment BME process.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the disclosure are achieved and other advantageous results attained.

As various changes could be made in the above products and methods without departing from the scope of the disclosure, it is intended that all matter contained in the above description and the associated drawings shall be interpreted as illustrative and not in a limiting sense.

1. A process for preparing iminodiacetic acid, the process comprising:
introducing a feed salt stream comprising disodium iminodiacetic acid (DSIDA) into a salt compartment of a two-compartment electrodialysis bipolar membrane cell comprising the salt compartment and a base compartment;
recovering a salt product stream from the salt compartment of the two-compartment bipolar membrane cell, the salt product stream comprising iminodiacetic acid (IDA) and monosodium iminodiacetic acid (MSIDA); and
recovering a base product stream from the base compartment of the two-compartment bipolar membrane cell, the base product stream comprising sodium hydroxide;
contacting the salt product stream with a crystallizer, thereby forming a crystallizer stream;
contacting the crystallizer stream with a filtration system, thereby forming a solid product stream comprising iminodiacetic acid and a recirculation stream;
wherein the recirculation stream is combined with the feed salt stream prior to introduction into the salt compartment of the two-compartment electrodialysis bipolar membrane cell; and
wherein at least a portion of the feed salt stream comprising DSIDA is prepared by reacting the base product stream comprising sodium hydroxide with diethanolamine (DEA) in the presence of a catalyst.

2. A process for preparing iminodiacetic acid, the process comprising:
introducing a feed salt stream comprising disodium iminodiacetic acid (DSIDA) into a salt compartment of a two-compartment electrodialysis bipolar membrane cell comprising the salt compartment and a base compartment;
recovering a salt product stream from the salt compartment of the two-compartment bipolar membrane cell, the salt product stream comprising iminodiacetic acid (IDA) and monosodium iminodiacetic acid (MSIDA); and
recovering a base product stream from the base compartment of the two-compartment bipolar membrane cell, the base product stream comprising sodium hydroxide;
contacting the salt product stream with a crystallizer, thereby forming a crystallizer stream;
contacting the crystallizer stream with a filtration system, thereby forming a solid product stream comprising IDA and a recirculation stream;
wherein the recirculation stream is combined with the feed salt stream prior to introduction into the salt compartment of the two-compartment electrodialysis bipolar membrane cell; and
wherein the process further comprises phosphonomethylating the IDA in the solid product stream.

3. A process for preparing iminodiacetic acid, the process comprising:
introducing a feed salt stream comprising disodium iminodiacetic acid (DSIDA) into a salt compartment of a two-compartment electrodialysis bipolar membrane cell comprising the salt compartment and a base compartment;
recovering a salt product stream from the salt compartment of the two-compartment bipolar membrane cell comprising the amino acid, the salt product stream comprising iminodiacetic acid (IDA) and monosodium iminodicacetic acid (MSIDA); and
recovering a base product stream from the base compartment of the two-compartment bipolar membrane cell, the base product stream comprising sodium hydroxide;
contacting the salt product stream with a crystallizer, thereby forming a crystallizer stream;
contacting the crystallizer stream with a filtration system, thereby forming a solid product stream comprising the iminodiacetic acid and a recirculation stream;
wherein the recirculation stream is combined with the feed salt stream prior to introduction into the salt compartment of the two-compartment electrodialysis bipolar membrane cell;
wherein at least a portion of the feed salt stream comprising DSIDA is prepared by reacting the base product stream comprising sodium hydroxide with diethanolamine in the presence of a catalyst to form DSIDA; and
wherein the process further comprises phosphonomethylating the iminodiacetic acid in the solid product stream.

4. A process for preparing iminodiacetic acid, the process comprising:
introducing a feed salt stream comprising disodium iminodiacetic acid (DSIDA) into a salt compartment of a two-compartment electrodialysis bipolar membrane cell comprising the salt compartment and a base compartment;
recovering a salt product stream from the salt compartment of the two-compartment bipolar membrane cell, the salt product stream comprising iminodiacetic acid (IDA) and monosodium iminodicacetic acid (MSIDA);
recovering a base product stream from the base compartment of the two-compartment bipolar membrane cell, the base product stream comprising sodium hydroxide; and
contacting the salt product stream with a crystallizer, thereby forming a crystallizer stream;
wherein the base product stream is essentially chloride (Cl⁻) free and at least a portion of the feed salt stream comprising DSIDA is prepared by reacting the base product stream comprising sodium hydroxide with diethanolamine (DEA) in the presence of a catalyst.

5. The process of claim 1 wherein the salt compartment of the two-compartment bipolar membrane cell is bounded by a bipolar membrane and a cation exchange membrane and the base compartment of the two-compartment bipolar membrane is bounded by the cation exchange membrane bounding the salt compartment and a second bipolar membrane.

6. The process of claim 1 wherein the two-compartment bipolar membrane cell further comprises an anode and a cathode.

7. The process of claim 6 further comprising applying an electric potential between the cathode and the anode of the two compartment bipolar membrane cell, thereby inducing flow of cations from DSIDA in the salt compartment through the cation exchange membrane into the base compartment of the two-compartment bipolar membrane cell.

8. The process of claim 1 wherein the current efficiency of the two-compartment bipolar membrane cell based on the transport of a cation of DSIDA to the base compartment is at least about 80%.

9. The process of claim 1 wherein the concentration of DSIDA in the feed salt stream is at least about 5 wt %.

10. (canceled)

11. The process of claim 1 wherein the salt product stream comprises from about 2 to about 20 wt % iminodiacetic acid.

12. The process of claim 1 wherein the salt product stream comprises at least about 5 wt. %, monosodium iminodiacetic acid.

13. The process of claim 1 wherein the salt product stream comprises less than about 30 wt. % monosodium iminodiacetic acid.

14. The process of claim 1 wherein the solid product stream comprises at least about 10 wt. % iminodiacetic acid.

15. The process of 1 wherein preparing the DSIDA by reacting the base product stream comprising sodium hydroxide with diethanolamine (DEA) in the presence of a catalyst comprises dehydrogenation of diethanolamine, wherein the dehydrogenation reaction comprises a strong base and a catalyst.

16. (canceled)

17. (canceled)

18. The process of claim 1 wherein preparing the DSIDA by reacting the base product stream comprising sodium hydroxide with diethanolamine (DEA) in the presence of a catalyst comprises catalytic oxidation of diethanolamine, wherein the catalyst is susceptible to poisoning or deactivation by chloride.

19. (canceled)

20. The process of claim 1 wherein the base product stream comprises at least about 5 wt. % of a base.

21. The process of claim 1 wherein the solid product stream is further processed to form an iminodiacetic acid wetcake, wherein the further processing comprises one or more of drying, evaporating, or heating.

22. (canceled)

23. The process of claim 21 wherein the iminodiacetic acid wetcake is used in a process for preparing N-(phosphonomethyl)iminodiacetic acid (PMIDA).

24. The process of claim 1 wherein the base product stream comprises NaOH and the specific power usage is less than about 3,000 kWhr/Ton base.

25. The process of claim 2, wherein phosphonomethylating the iminodacetic acid in the solid product stream forms N-(phosphonomethyl)iminodiacetic acid (PMIDA) or a salt thereof, wherein at least a portion of the PMIDA or salt thereof is used in a process for preparing N-(phosphonomethyl)glycine (glyphosate).

26. (canceled)
